(19) United States
(12) Patent Application Publication
(10) Pub. No.: US 2026/0263834 A1
(43) Pub. Date: Sep. 10, 2026

(54) INTRACAVITARY APPLICATORS, ASSEMBLIES, AND KITS FOR RADIATION ONCOLOGY TREATMENT

(71) Applicant: University of Kentucky Research Foundation, Lexington, KY (US)

(72) Inventors: Ali Soleimani-Meigooni, Lexington, KY (US); Ellis L. Johnson, Lexington, KY (US); Denise Fabian, Lexington, KY (US)

(21) Appl. No.: 19/559,436

(22) Filed: Mar. 6, 2026

Related U.S. Application Data
(60) Provisional application No. 63/768,084, filed on Mar. 6, 2025.
(60) Provisional application No. 63/768,295, filed on Mar. 7, 2025.

(51) Int. Cl. A61N 5/10

(52) U.S. Cl. CPC A61N 5/1016; A61N 2005/1018; A61N 2005/1024

(57) ABSTRACT

An intracavitary applicator for radiation oncology can include an applicator body configured to be inserted in a vaginal fornix of an individual. The applicator body can include a longitudinal channel configured to receive a conduit that receives and guides a radiation source, and a lateral channel providing passage from an exterior of the applicator body to the longitudinal channel. A fastener can be movably received in the lateral channel such that the fastener can be selectively manipulated in a first manner to maintain the conduit in association with the applicator body, and in a second manner to permit removal of the conduit. The applicator body can further include one or more additional lateral channels configured to receive and guide a brachytherapy needle through the applicator body. The intracavitary applicator can be utilized in intracavitary applicator assemblies and kits for radiation oncology.

### RELATED APPLICATIONS

This application claims priority to U.S. Patent Application Ser. No. 63/768,084 filed on Mar. 6, 2025 and U.S. Patent Application Ser. No. 63/768,295, filed on Mar. 7, 2025 the entire disclosure of each of which is incorporated herein by reference.

### TECHNICAL FIELD

The present disclosure generally relates to intracavitary applicators, assemblies, and kits for radiation oncology treatment. In particular, certain embodiments of the present disclosure relate to applicators, assemblies, and kits that include an applicator body that can be selectively secured to a conduit for guiding a radiation source into the applicator body and/or used as a guide for one or more brachytherapy needles during brachytherapy treatment.

### INTRODUCTION

Brachytherapy is a treatment used to treat various types of cancers, including vaginal, cervical, uterine, and endometrial cancer. During brachytherapy, a radioactive source (or radiation source) is placed inside an applicator that is implanted in the body of the patient in proximity to a tumor or an area from which a tumor was excised. Intracavitary placement of the radiation source during brachytherapy generally occurs around a natural body cavity. This technique facilitates a more targeted and concentrated dose of radiation to the cancer cells while minimizing damage to surrounding healthy tissues relative to the alternative method of treatment with external beam radiation. Brachytherapy can be utilized alone or in combination with other cancer treatment techniques, such as external beam radiotherapy and chemotherapy. Based on the location and size of the tumor, different types of applicators have been designed and gradually modified to improve quality treatments.

Locally advanced cervical cancer is sometimes treated utilizing a combination of external beam radiation therapy and brachytherapy. Adequate dosing of the cervical tumor with brachytherapy is essential to achieve good tumor control. Applicators used for such applications include tandem and ovoid applicators, tandem and ring applicators, and hybrid applicators which include an intracavitary component and an interstitial component that allows for the insertion of a radiation source into cancerous tissue.

Tandem and ovoid applicators of known construction, however, often fail to deliver optimal radiation to high-risk clinical target volumes (HRCTVs) in the cervix due to the residual disease burden being asymmetrical or too large. This is especially true in instances where the patient has a bulky cervix where one or more portions of the HRCTV often falls outside the range of the 100 percent isodose line of the applicator. Utilizing needles provided with an applicator known as a hybrid template applicator can help to enhance the radiation dose distribution by providing additional pathways for a radiation source to be placed in areas of the tumor. However, hybrid template applicators of known construction have a major issue: the needles with metal stylets have difficulty in bending and going through at desired insertion angles. Therefore, patient treatment is not optimal. In addition, these applicators are generally very expensive and require purchase of an entire device in order to be utilized, which makes it difficult for many therapeutic centers to implement a hybrid applicator for their treatments.

### SUMMARY

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The present disclosure includes intracavitary applicators, assemblies, and kits useful in radiation oncology applications, such as brachytherapy treatment. In particular, the present disclosure includes intracavitary applicators, assemblies, and kits that include an applicator body that can be selectively secured to a conduit for guiding a radiation source into the applicator body and/or used as a guide for one or more brachytherapy needles during brachytherapy treatment.

An exemplary intracavitary applicator can include an applicator body configured to be inserted in a vaginal fornix of an individual. The applicator body can include a longitudinal channel configured to receive a conduit that receives and guides a radiation source, and a lateral channel providing passage from an exterior of the applicator body to the longitudinal channel. A fastener can be movably received in the lateral channel such that the fastener can be selectively manipulated in a first manner to maintain the conduit in association with the applicator body when the conduit is received in the longitudinal channel, and in a second manner to permit removal of the conduit from the longitudinal channel. In some embodiments, the fastener is removably received in the lateral channel, such that the applicator body and the fastener can be separated from each other. In some embodiments, an interior surface defining the lateral channel and an exterior surface of the fastener include compatible threading to allow positional adjustment of the fastener by rotation. The applicator body can include one or more curved surfaces.

The applicator body can further include one or more additional lateral channels configured to receive and guide a brachytherapy needle through the applicator body. In some implementations, one or more brachytherapy needles can be inserted through the one or more additional lateral channels to provide a pathway for a radiation source to apply radiation dosing to anatomical areas that do not receive radiation dosing when the radiation source is deposited in the longitudinal channel of the applicator body. A centerline of an additional lateral channel can be angled relative to at least one of: (i) a horizontal plane bisecting a first open end of the additional lateral channel and (ii) a vertical plane along which a longitudinal axis of the applicator body extends. In some embodiments, the centerline of an additional lateral channel intersects the horizontal plane bisecting the first open end of the additional lateral channel and/or the vertical plane along which the longitudinal axis of the applicator body extends at an angle of about 0.1 degree to about 15 degrees. In some embodiments, there are two or more additional lateral channels configured to receive and guide a brachytherapy needle. In some embodiments, each of the two or more additional lateral channels is located on the same side of the vertical plane along which the longitudinal axis of the applicator body extends.

An exemplary intracavitary applicator assembly can include: a first intracavitary applicator; a first conduit removably secured to the first intracavitary applicator and configured to receive and guide a radiation source; a second intracavitary applicator; and a second conduit removably secured to the second intracavitary applicator and configured to receive and guide the radiation source. The first intracavitary applicator includes a first applicator body with a longitudinal channel and a lateral channel, and a first fastener movably received in the lateral channel. The second intracavitary applicator includes a second applicator body with a longitudinal channel and a lateral channel, and a second fastener movably received in the lateral channel. The first applicator body and the second applicator body can each be configured for insertion into a vaginal fornix of an individual. The first applicator body and/or second applicator body can further include an additional lateral channel for guiding a brachytherapy needle. The centerline of the additional lateral channel can be angled. The intracavitary applicator assembly can optionally include one or more of: a tandem applicator configured to be inserted into a cervical canal and to receive and guide the radiation source; a template including a plurality of openings to selectively receive brachytherapy needles; and a locking device configured to be selectively manipulated to hold the first conduit and the second conduit in a fixed position.

An exemplary intracavitary applicator kit can include: a conduit for receiving and guiding a radiation source; a first intracavitary applicator configured to be removably secured to the conduit; and a second intracavitary applicator configured to be removably secured to the conduit. The first intracavitary applicator includes a first applicator body with a longitudinal channel for receiving the conduit and a lateral channel, and a first fastener. The first fastener is movably received in the lateral channel of the first applicator body, such that the first fastener can be selectively manipulated to maintain or release the conduit from the longitudinal channel of the first applicator body. The second intracavitary applicator includes a second applicator body with a longitudinal channel for receiving the conduit and a lateral channel, and a second fastener. The second fastener is movably received in the lateral channel of the second applicator body, such that the second fastener can be selectively manipulated to maintain or release the conduit from the longitudinal channel of the second applicator body. The first applicator body and the second applicator body can each be configured for insertion into a vaginal fornix of an individual. In some embodiments, a diameter of the first applicator body is different from a diameter of the second applicator body. The first applicator body and/or the second applicator body can include one or more additional lateral channels configured to receive and guide a brachytherapy needle through the body. The number of additional lateral channels, orientation of additional lateral channels, and/or angle of additional lateral channels can differ between the first applicator body and the second applicator body.

### BRIEF DESCRIPTION OF THE DRAWINGS

FIG. 1 is a perspective view of an intracavitary applicator assembly including an intracavitary applicator made in accordance with an embodiment of present disclosure, with a brachytherapy needle inserted through the intracavitary applicator;

FIG. 2 is another perspective view of the intracavitary applicator assembly of FIG. 1, but with a conduit of the intracavitary applicator assembly removed from the intracavitary applicator;

FIG. 3 is a top view of the intracavitary applicator assembly of FIG. 1;

FIG. 4 is a cross-sectional view of the intracavitary applicator assembly taken along 4-4 in FIG. 3;

FIG. 5 is a perspective view of the intracavitary applicator of FIG. 1;

FIG. 6 is a side view of the intracavitary applicator of FIG. 1;

FIG. 7 is a cross-sectional view of the intracavitary applicator taken along 7-7 in FIG. 6;

FIG. 8 is a cross-sectional view of the intracavitary applicator taken along 8-8 in FIG. 6;

FIG. 9 is a perspective view of another intracavitary applicator made in accordance with an embodiment of the present disclosure;

FIG. 10 is a perspective view of another intracavitary applicator made in accordance with an embodiment of the present disclosure;

FIG. 11 is a side view of the intracavitary applicator of FIG. 10;

FIG. 12 is a cross-sectional view of the intracavitary applicator taken along 12-12 in FIG. 11;

FIG. 13 is a perspective view of another intracavitary applicator made in accordance with an embodiment of the present disclosure; and

FIG. 14 is a perspective view of another intracavitary applicator assembly made in accordance with an embodiment of the present disclosure, with a first brachytherapy needle and a second brachytherapy needle provided therewith.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly, the specific details of the described exemplary embodiments, is provided primarily for clearness of understanding and no unnecessary limitations are to be understood therefrom. In case of conflict, the specification of this document, including definitions, will control.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Unless otherwise indicated, all numbers expressing quantities of dimension and angle used in the specification are to be understood as being modified in all instances by the term “about”. Accordingly, unless indicated to the contrary, the numerical parameters set forth in this specification are approximations that can vary depending upon the desired properties sought to be obtained by the presently disclosed subject matter.

As used herein, the term “about,” when referring to a value or to dimension or angle is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

It is understood that where a range of units is disclosed, such range is inclusive of the starting and end units. For example, if a range of “10 to 15” or “between 10 to 15” is disclosed, then 10 and 15 are considered part of such range, unless stated otherwise. It is also understood that each unit between two particular units are also disclosed. For example, if 10 and 15 are disclosed, then 11, 12, 13, and 14 are also disclosed.

The presently disclosed subject matter includes intracavitary applicators, assemblies, and kits for radiation oncology. As will be appreciated by one of ordinary skill in the art upon study of this document, the intracavitary applicators, assemblies, and kits disclosed herein can be useful during brachytherapy treatment of an individual, such as a human born biologically female. Cancers for which brachytherapy treatment may be applied to treat include, by way of non-limiting example, vaginal, cervical, uterine, and endometrial cancer.

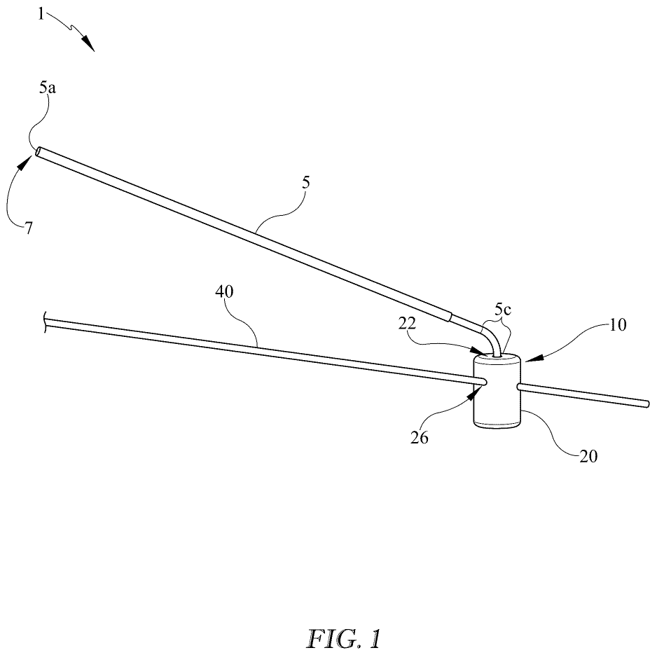

Referring first to FIGS. 1-4, an intracavitary applicator assembly (or assembly) 1 in accordance with an embodiment of the present disclosure is provided and includes: a conduit 5; and an intracavitary applicator (or applicator) 10, which can be removably secured to the conduit 5. The conduit 5 is configured to receive and guide a radiation source (not shown) into the applicator 10 when the two are removably secured together and the assembly 1 is in use. To this end, the conduit 5 includes an open proximal end 5a, a closed distal end 5b, and a channel 7 that substantially extends the length of the conduit 5. Alternative embodiments in which the distal end 5b of the of the conduit 5 is open are, however, also contemplated herein. As shown, in this embodiment, the conduit 5 is of the same general construction of radiation source conduits known within the art and commonly coupled with brachytherapy ovoid applicators of known construction. In this regard, and in this embodiment, the conduit 5 includes a curved portion 5c that causes the distal end 5b of the conduit to be angled relative to the main body of the conduit 5, which extends from the proximal end 5a to the curved portion 5c of the conduit 5.

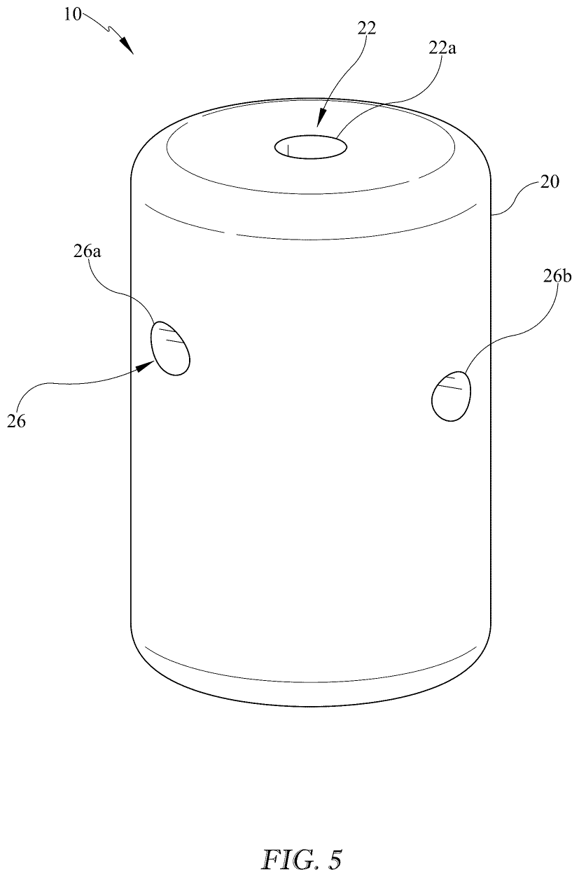

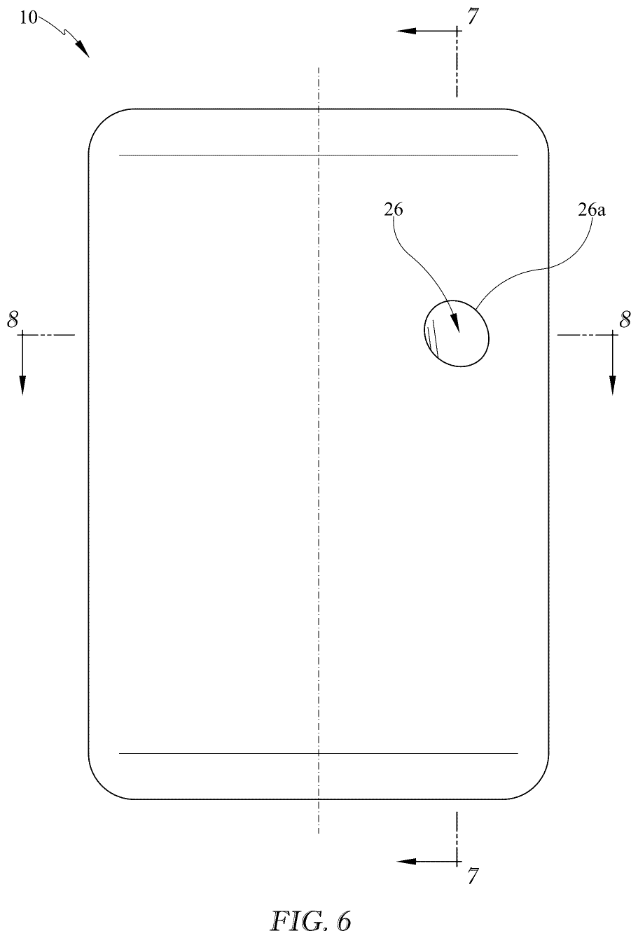

Referring now to FIGS. 1-8, the applicator 10 includes an applicator body 20 and a fastener 30 that is movably received in the applicator body 20. The fastener 30 can be selectively manipulated to secure the conduit 5 to, and release the conduit 5 from, the applicator body 20. To facilitate insertion in the vaginal fornix of an individual, the applicator body 20 preferably includes one or more rounded surfaces. In this exemplary embodiment, the applicator body 20 is substantially cylindrically shaped and includes a top surface, a bottom surface, a rounded side wall, and rounded edges leading from the top and bottom surfaces to the rounded side wall. It should be appreciated, however, that the applicator body 20 is not necessarily limited to such construction and can be alternatively shaped without departing from the spirit and scope of the present disclosure. For instance, in some embodiments, the applicator body 20 may be ovoid shaped. In some embodiments, the top surface of the applicator body 20 may include a slight depression for guiding the distal end 5b of the conduit 5 into the applicator body 20.

Referring still to FIGS. 1-8, the applicator body 20, in this exemplary embodiment, includes a longitudinal channel 22 that, in this embodiment, includes an open proximal end 22a and an open distal end 22b. The longitudinal channel 22 is shaped and sized to receive the conduit 5, and thus the radiation source guided in the conduit 5. The applicator body 20 also includes a first lateral channel 24 in which the fastener 30 is movably received. To facilitate engagement of the fastener 30 with the conduit 5 while present in the longitudinal channel 22, the first lateral channel 24 provides passage from an exterior of the applicator body 20 to the longitudinal channel 22. In this regard, the first lateral channel 24 has a first open end 24a located at a first side portion of the applicator body 20 and a second open end 24b providing access to the longitudinal channel 22. It is appreciated that the first lateral channel 30 can, in various embodiments, be positioned at different locations about the applicator body 20 relative to that illustrated in the drawings without departing from the spirit and scope of the present disclosure.

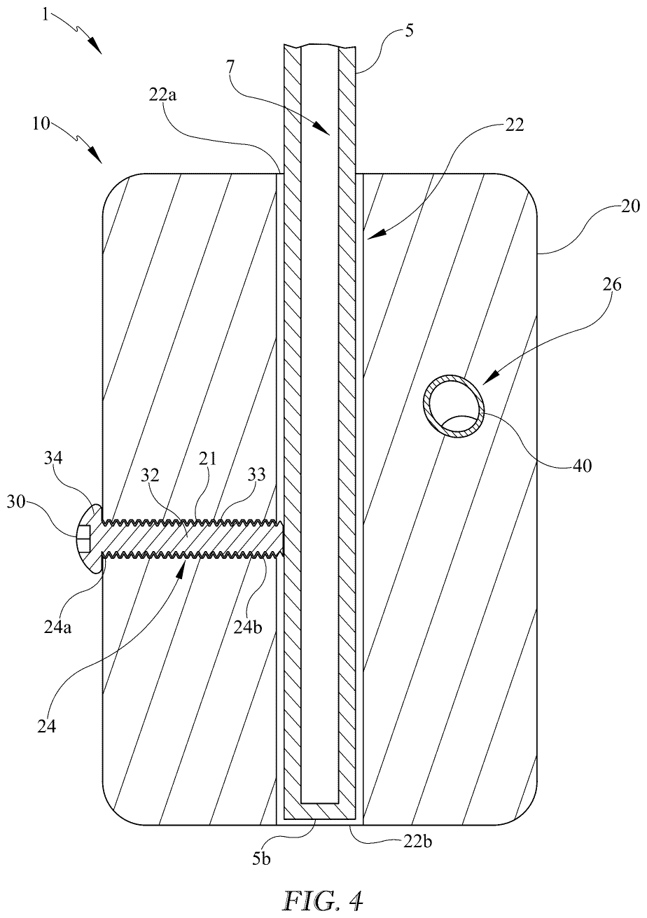

Referring now specifically to FIG. 4, to permit travel of the fastener 30 in the first lateral channel 24 while also ensuring the fastener 30 is not inadvertently dislodged from the first lateral channel 24, in this embodiment, a threaded connection is provided between the fastener 30 and the applicator body 20. In this regard, the interior surface 21 of the applicator body 20 defining the first lateral channel 24 is threaded, and an exterior surface of the main body 32 of the fastener 30 is threaded with compatible threading 33. When the conduit 5 is deposited in the longitudinal channel 22, the fastener 30 can be manipulated in a first manner while deposited in the first lateral channel 24 by rotating the fastener 30 in a first rotational direction (e.g., clockwise) to move the fastener 30 toward the conduit 5. The fastener 30 can thus be rotated in the first rotational direction to apply a clamping force to the conduit 5 that maintains the conduit 5 and the applicator body 20 in association with each other while the assembly 1 is in use, as best shown in FIG. 4. Conversely, the fastener 30 can be manipulated in a second manner while deposited in the first lateral channel 24 by rotating the fastener 30 in a second rotational direction (e.g., counterclockwise) to disengage and/or reduce the clamping force imposed upon the conduit 5 so that the conduit 5 and the applicator body 20 can be separated from each other by removing the conduit 5 from the longitudinal channel 22 of the applicator body 20.

Accordingly, unlike brachytherapy ovoid applicators of traditional construction, where the ovoid applicator body is permanently secured to the conduit that guides the radiation source into the ovoid applicator body, the applicator body 20, and thus applicator 10 as a whole, can be selectively and removably secured to a conduit for delivering a radiation source. As a result, the applicator 10 may, in some implementations be utilized in existing brachytherapy systems and with radiation source conduits. In this regard, and in some embodiments, the conduit 5 may be an existing conduit that the applicator 10 is retrofitted to. The use of applicators that can be removably secured to a radiation source conduit can also serve to increase the flexibility of an existing brachytherapy treatment system and ability for physicians to tailor treatment to better accommodate variation in individual anatomies by enabling a single radiation source conduit to be utilized with multiple different applicators, as will be further evident by the discussion that follows concerning intracavitary applicator kits made in accordance with the present disclosure.

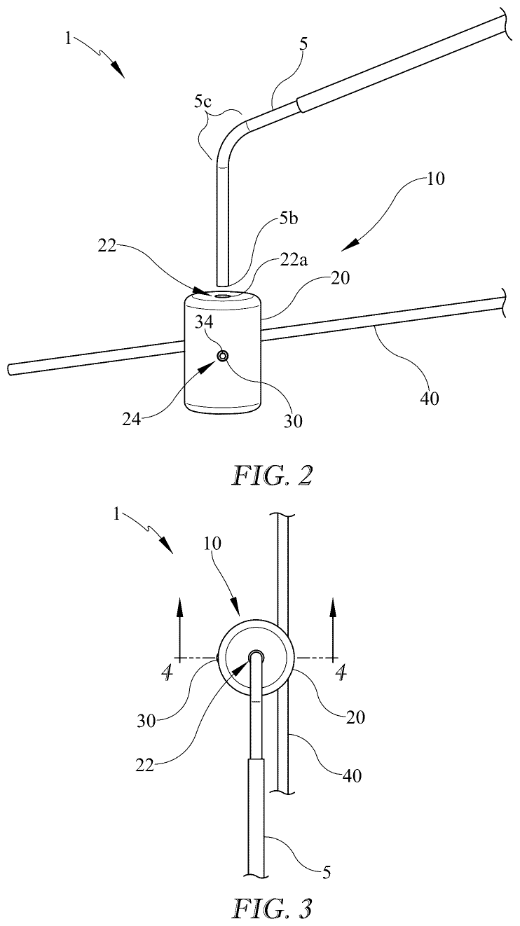

Referring now specifically to FIGS. 2-4, 6, and 8, in this exemplary embodiment, the fastener 30 can be fully removed from the applicator body by rotating the fastener 30 in the second rotational direction until the fastener 30 is dislodged from the first lateral channel 24. As such, in addition to being movably received in the first lateral channel 24, the fastener 30 in this exemplary embodiment is also removably received in the first lateral channel 24. As shown, in this exemplary embodiment, the fastener 30 includes a head 34 that is larger than the first open end 24a of the first lateral channel 24 and the fastener 30 has a length that is greater than the length of the first lateral channel 24 so that the fastener 30 can be manipulated to apply a clamping force upon the conduit 5. Alternative embodiments, are however contemplated in which the fastener 30 does not include a head 34 that is larger than the first open end 24a of the first lateral channel 24 and the length of the fastener 30 is smaller than the length of the first lateral channel 24. In such embodiments, a tool (e.g., a screwdriver or hex wrench) may be deposited into the first lateral channel 24 to engage the fastener 30 and manipulate the fastener 30.

Referring now to FIGS. 1-8, in this exemplary embodiment, the applicator body 20 further includes an additional, second lateral channel 26 that is configured to receive and guide a brachytherapy needle 40 through the applicator body 20, as shown in FIGS. 1-3. In this regard, the second lateral channel 26 has a first open end 26a located at a second side portion of the applicator body 20 and a second open end 26b located at a third side portion of the applicator body 20 so that the brachytherapy needle 40 can pass entirely through the applicator body 20. Accordingly, in this exemplary embodiment, the applicator body 20 provides a template for brachytherapy needle guidance that facilitates needle positioning to deliver radiation to a target area of an individual that may not receive radiation when the radiation source is deposited in the longitudinal channel 22 and the assembly 1 is in use. In some embodiments and implementations, the applicator body 20 may be rotated relative to that shown in FIGS. 1-4 and secured to the conduit 5 to affect the orientation of the second lateral channel 26 relative to the conduit 5.

In use, the assembly 1 can be utilized to apply brachytherapy treatment for cervical cancer by depositing the applicator body 20, while the applicator 10 is secured to the conduit 5, into a vaginal fornix of an individual. Once the applicator body 20 is deposited in the vaginal fornix, a radiation source can be selectively inserted into the channel 7 and guided to a position in the longitudinal channel 22 of the applicator body 20. In cases where it is desirable for radiation to be applied to additional anatomical areas than facilitated by deposit of the radiation source in the longitudinal channel 22 of the applicator body 20, the applicator 10 can be loaded with a brachytherapy needle 40 by inserting the brachytherapy needle 40 through the second lateral channel 26. In some cases, a metal stylet may be deposited in the brachytherapy needle 40 to provide rigidity to and accurate placement of the brachytherapy needle 40. Once the brachytherapy needle is provided in the desired orientation in the individual, the metal stylet can be removed and the radiation source selectively inserted into the brachytherapy needle 40.

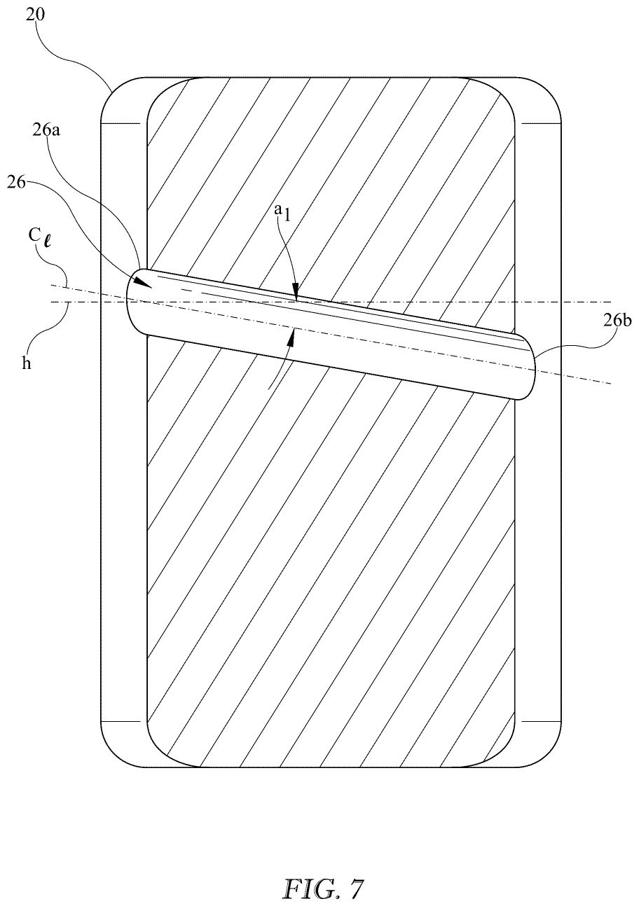

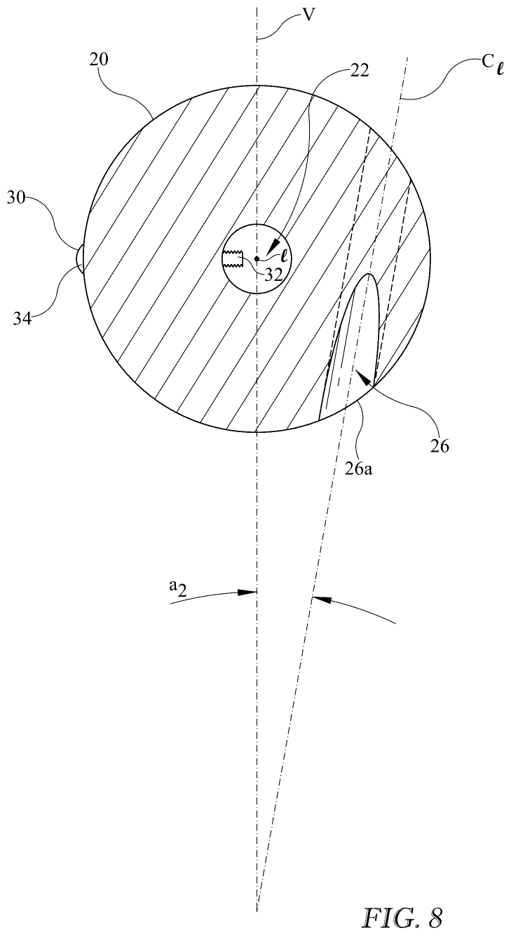

Referring now specifically to FIGS. 1-3, 7, and 8, the second lateral channel 26 can be angled to ease the insertion of the brachytherapy needle 40 and/or to facilitate placement of the brachytherapy needle 40 at the desired location for a target treatment area in an individual. The second lateral channel 26 can be vertically angled, such that a brachytherapy needle 40 inserted therethrough slopes downwardly or upwardly when the applicator body 20 is in an upright position, as best shown in FIGS. 1 and 2. The second lateral channel 26 can additionally or alternatively be laterally angled, such that a brachytherapy needle 40 moves in a left-to-right or right-to-left manner as it extends through the second lateral channel 26 when the applicator body 20 is in an upright position, as show best in FIG. 3. In this exemplary embodiment, the second lateral channel 26 is both vertically and laterally angled. The degree to which the second lateral channel 26 is angled, either outwardly or downwardly, can be adapted to accommodate different brachytherapy applications, accommodate different individual anatomies, and/or facilitate brachytherapy needle 40 placement in different treatment areas. In some embodiments, the second lateral channel 26 is vertically angled, such that a centerline, cₗ, of the second lateral channel 26 intersects with a horizontal plane, h, bisecting the first open end 26a of the second lateral channel 26 at an angle, a₁, as shown best in FIG. 7. In some embodiments, the centerline, cₗ, of the second lateral channel 26 intersects with the horizontal plane, h, bisecting the first open end 26a of the second lateral channel 26 at an angle, a₁, ranging from about 0.1 degree to about 15 degrees. In some embodiments, the second lateral channel 26 is laterally angled, such that a centerline, c₁, of the second lateral channel 26 intersects with a vertical plane, v, along which the longitudinal axis, l, at an angle, a₂, as shown best in FIG. 8. In some embodiments, the second lateral channel 26 is laterally angled, such that the centerline, c₁, of the second lateral channel 26 intersects with a vertical plane, v, along which the longitudinal axis, l, of the applicator body 20 extends at an angle, a₂, ranging from about 0.1 degree to about 15 degrees.

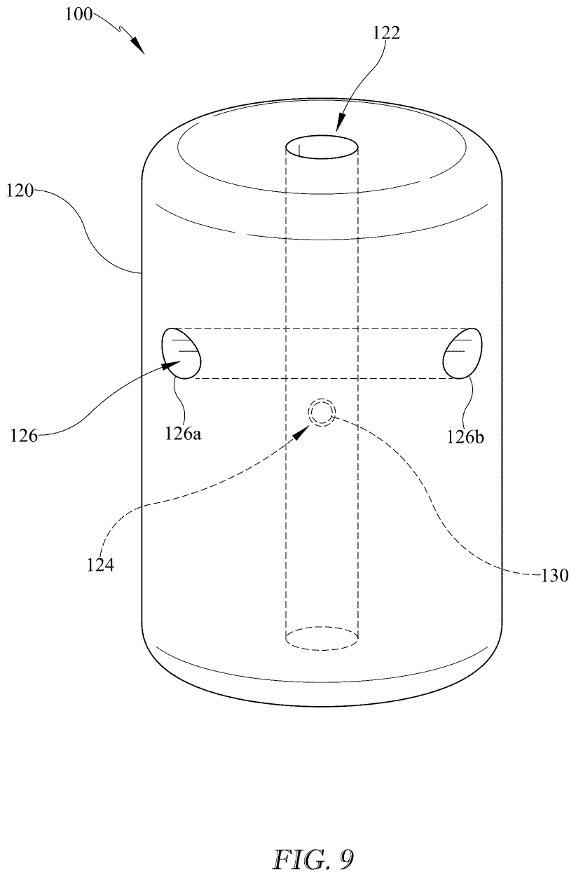

Referring now to FIG. 9, another exemplary embodiment of an intracavitary applicator (or applicator) 100 is provided. The applicator 100 in this embodiment is of the same construction and functions in the same manner as the applicator 10 described above with FIGS. 1-8, except as otherwise noted. In this regard, the applicator 100 also includes: an applicator body 120 with a longitudinal channel 122, a first lateral channel 124, and a second lateral channel 126; and a fastener 130 movably received in the first lateral channel 124. However, in this exemplary embodiment, the second lateral channel 126 is neither vertically nor laterally angled. As a result, the first open end 126a and the second open end 126b of the second lateral channel 126 are positioned directly opposite of each other. Accordingly, a brachytherapy needle inserted through the second lateral channel 126 in this embodiment will neither slope upwardly or downwardly nor move in either a left-to-right or right-to-left manner as it extends through the second lateral channel 126.

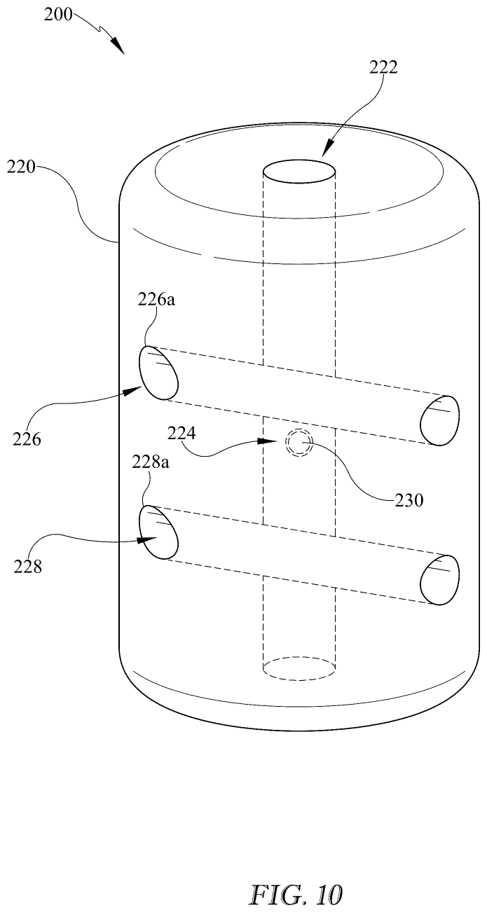

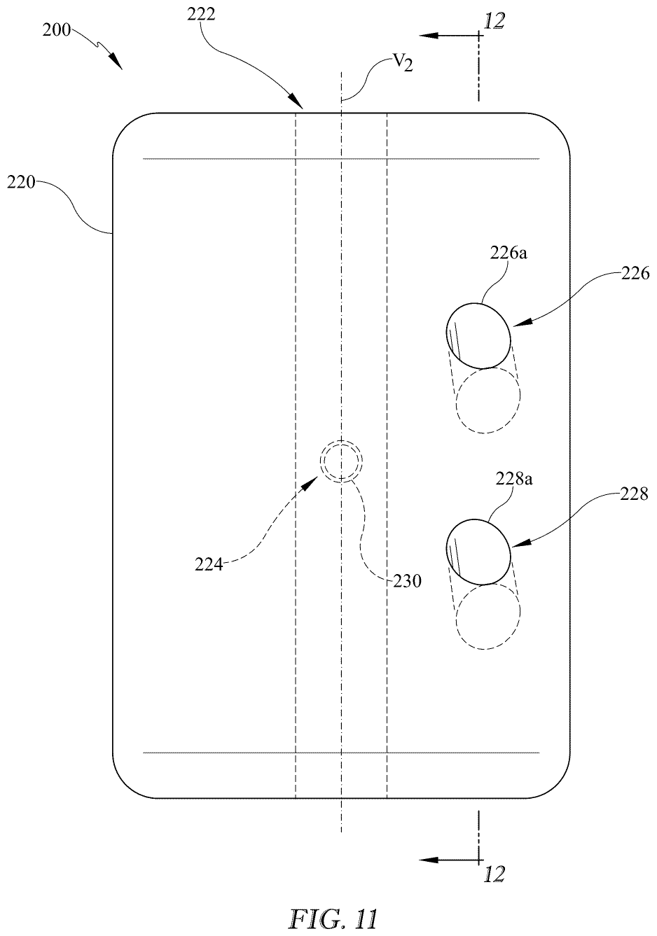

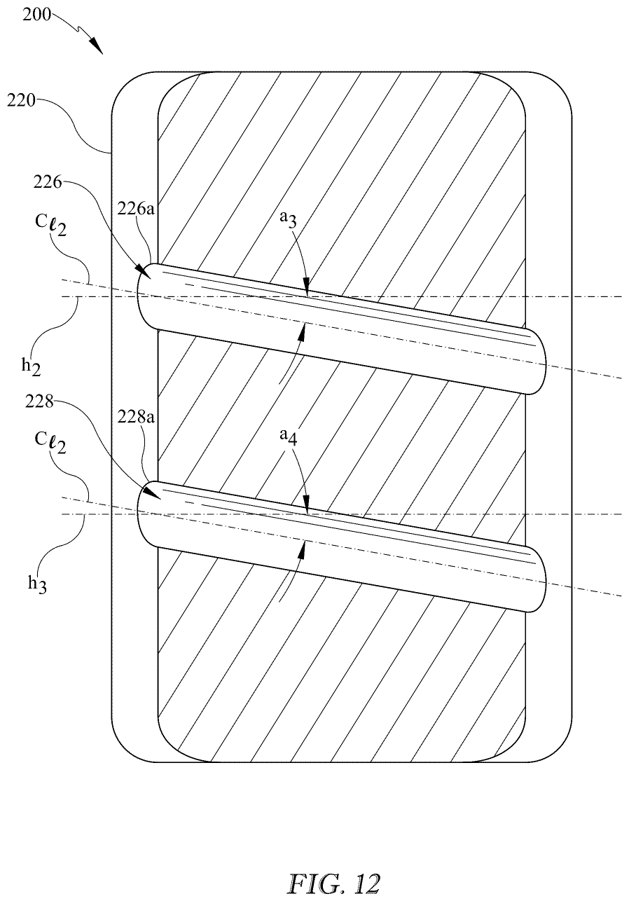

Referring now to FIGS. 10-12, another exemplary embodiment of an intracavitary applicator (or applicator) 200 is provided. The applicator 200 in this embodiment is of the same construction and functions in the same manner as the applicator 10 described above with FIGS. 1-8, except as otherwise noted. In this regard, the applicator 200 also includes: an applicator body 220 with a longitudinal channel 222, a first lateral channel 224, and a second lateral channel 226; and a fastener 230 movably received in the first lateral channel 224. However, in this exemplary embodiment, the applicator body 220 further includes a third lateral channel 228 through which a brachytherapy needle can be passed. Accordingly, in this exemplary embodiment, the applicator 200 can serve as a guide for multiple brachytherapy needles. The third lateral channel 228 is positioned so that both the second lateral channel 226 and the third lateral channel 228 are positioned on the same side, in this case, right side, of a vertical plane, v₂, along which a longitudinal axis of the applicator body 220 extends, as shown best in FIG. 11. In this embodiment, the third lateral channel 228 is positioned directly below and in parallel with the second lateral channel 226.

Referring still to FIGS. 10-12, in this exemplary embodiment, the second lateral channel 226 and the third lateral channel 228 are both angled in the same manner as the second lateral channel 26 of the applicator described above with reference to FIGS. 1-8. In this regard, the second lateral channel 226 and the third lateral channel 228 are both vertically angled, such that a centerline, cₗ₂, cₗ₃, of the second and third lateral channels 226, 228 intersects with a horizontal plane, h₂, h₃, bisecting the first open ends 226a, 228a of the second lateral channel 226 at an angle, a₃, a₄. In some embodiments, angles a₃ and a₄ range from about 0.1 degree to about 15 degrees. In this exemplary embodiment, the second lateral channel 226 and the third lateral channel 228 are both also laterally angled, such that the centerline, cₗ₂, cₗ₃, of the second lateral channel 226 and the third lateral channel 228 intersects with a vertical plane along which the longitudinal axis of the applicator body 220 extends at an angle. In some embodiments, the centerline, cₗ₂, cₗ₃, of the second lateral channel 226 and the third lateral channel 228 intersects with the vertical plane along which the longitudinal axis of the applicator body 220 extends at an angle ranging from about 0.1 degree to about 15 degrees.

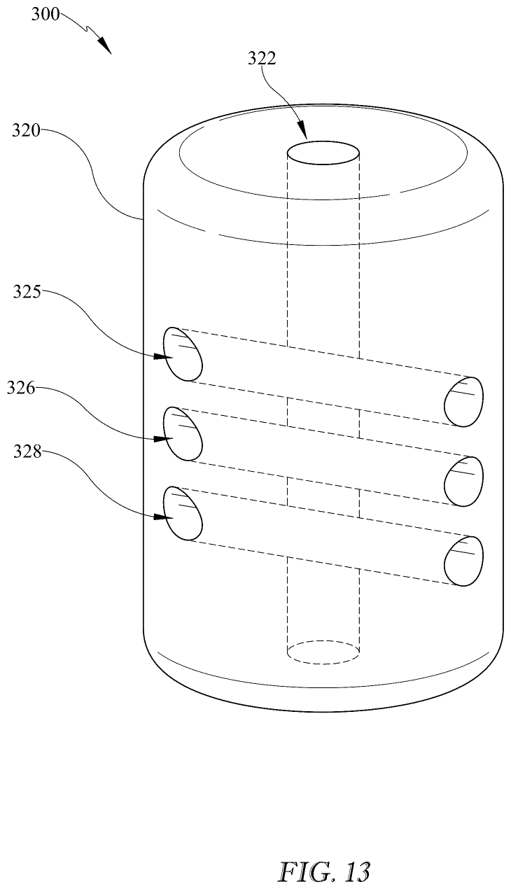

Referring now to FIG. 13, another exemplary embodiment of an intracavitary applicator (or applicator) 300 is provided. The applicator 300 in this embodiment is of the same construction and functions in the same manner as the applicator 200 described above with FIGS. 10-12, except as otherwise noted. In this regard, the applicator 300 also includes: an applicator body 320 with a longitudinal channel 322, a first lateral channel (not shown), a second lateral channel 325, and a third lateral channel 326; and a fastener (not shown) movably received in the first lateral channel. However, in this exemplary embodiment, the applicator body 320 further includes a fourth lateral channel 328 through which a brachytherapy needle can be passed. In this embodiment, the fourth lateral channel 328 is positioned directly below and in parallel with the third lateral channel 326.

Although the applicators 200, 300 with multiple lateral channels 226, 228, 325, 326, 328 for receiving a brachytherapy needle are primarily described above in the context of being both vertically and laterally oriented, the applicators 200, 300 are not limited to such construction. Rather, in some alternative embodiments, some or all of the lateral channels 226, 228, 325, 326, 328 for receiving a brachytherapy needle may be laterally angled but not vertically angled, or vice versa. In some alternative embodiments, some or all of the lateral channels 226, 228, 325, 326, 328 for receiving a brachytherapy needle may be neither vertically angled nor laterally angled, like that of the second lateral channel 126 of the applicator 100 described above with reference to FIG. 9. In some alternative embodiments, the lateral channels 226, 228, 325, 326, 328 for receiving a brachytherapy needle include a combination of lateral channels that are vertically and/or laterally angled and lateral channels that are neither vertically nor laterally angled. The degree to which different lateral channels 226, 228, 325, 326, 328 for receiving a brachytherapy needle are angled, vertically and/or laterally, can differ from each other.

Applicators with different dimensions and features can be constructed in accordance with the present disclosure to accommodate different target treatment areas and/or the anatomies of a wide range of individuals. For instance, in various embodiments, the diameter of the applicator body 20, 120, 220, 320 of the applicators 10, 100, 200, 300 may range from about 2.0 centimeters (cm) to about 3.5 cm. In various embodiments, the radial distance of the lateral channels 26, 126, 226, 228, 325, 326, 328 for receiving a brachytherapy needle of the applicators 10, 100, 200, 300 from the center of the applicator body 20, 120, 220, 320 may range from about 0.5 cm to about 1.2 cm. In various embodiments, where the applicator body includes only a single lateral channel for receiving a brachytherapy needle, the longitudinal distance between the lateral channel for receiving the brachytherapy needle and a midpoint of the applicator body may range from about 1.0 cm below the midpoint (−1.0 cm) to about 1.0 cm above the midpoint (+1.0 cm). In some embodiments, where the applicator body includes two lateral channels for receiving a brachytherapy needle, the longitudinal distance between a first lateral channel for receiving the brachytherapy needle and a midpoint of the applicator body may be about 1.0 cm below the midpoint (−1.0 cm) and between a second lateral channel for receiving the brachytherapy needle and the midpoint may be about 1.0 cm above the midpoint (+1.0 cm). In some embodiments, where the applicator body includes three lateral channels for receiving a brachytherapy needle, the longitudinal distance between: a first lateral channel for receiving the brachytherapy needle and a midpoint of the applicator body may be about 1.0 cm below the midpoint (−1.0 cm); between a second lateral channel for receiving the brachytherapy needle and the midpoint may be 0.0 cm (i.e., provided at the midpoint); and between a third lateral channel for receiving the brachytherapy needle and the midpoint may be 1.0 cm above the midpoint (+1.0 cm).

As evidenced, for example, in TABLES 1-4 below, various embodiments of the applicators 10, 100, 200, 300 can be established by modifying: the diameter of the applicator body 20, 120, 220, 320, the number of lateral channels 26, 126, 226, 228 325, 326, 328 for receiving a brachytherapy needle; degree to which the channels 26, 26, 226, 228, 325, 326, 328 are laterally angled; the radial distance between the lateral channels 26, 126, 226, 228 325, 326, 328 and the center of the applicator body 20, 120, 220, 320; and/or the longitudinal distance between a midpoint of the applicator body 20, 120, 220, 320 and the lateral channels 26, 126, 226, 228, 325, 326, 328. Various embodiments can, in addition to or as alternative to being laterally angled, also be established by modifying the degree to which the channels 26, 26, 226, 228, 325, 326, 328 are vertically angled.

TABLE 1
Applicator embodiments with two lateral channels for receiving a
brachytherapy needle, each lateral channel for receiving brachytherapy
needle having either 0 degree or 10 degree lateral angle.

| | | | Total | | Longi- |
|---|---|---|---|---|---|
| | | Lateral | Number of | Radial | tudinal |
| | Diameter | Angle of | Lateral | Distance | distance |
| | of the | Channel for | Channels | From the | from the |
| | Applicator | Receiving | for | center of | middle of |
| Applicator | Body | Needle (a₂) | Receiving | the ovoid | Ovoid |
| # | (cm) | (degrees) | Needle | (cm) | (cm) |
| 1A | 2 | 0 | 2 | 0.5 | −1.0, +1.0 |
| 2A | 2 | 10 | 2 | 0.5 | −1.0, +1.0 |
| 3A | 2.5 | 0 | 2 | 0.7 | −1.0, +1.0 |
| 4A | 2.5 | 10 | 2 | 0.7 | −1.0, +1.0 |
| 5A | 3 | 0 | 2 | 1.0 | −1.0, +1.0 |
| 6A | 3 | 10 | 2 | 1.0 | −1.0, +1.0 |
| 7A | 3.5 | 0 | 2 | 1.2 | −1.0, +1.0 |
| 8A | 3.5 | 10 | 2 | 1.2 | −1.0, +1.0 |

TABLE 2
Applicator embodiments with three lateral channels for receiving
a brachytherapy needle, each lateral channel for receiving brachytherapy
needle having either 0 degree or 10 degree lateral angle.

| | | | Total | | Longi- |
|---|---|---|---|---|---|
| | | Lateral | Number of | Radial | tudinal |
| | Diameter | Angle of | Lateral | Distance | distance |
| | of the | Channel for | Channels | From the | from the |
| | Applicator | Receiving | for | center of | middle of |
| Applicator | Body | Needle (a₂) | Receiving | the ovoid | Ovoid |
| # | (cm) | (degrees) | Needle | (cm) | (cm) |
| 1B | 2 | 0 | 3 | 0.5 | −0.5, |
| | | | | | 0, +0.5 |
| 2B | 2 | 10 | 3 | 0.5 | −0.5, |
| | | | | | 0, +0.5 |
| 3B | 2.5 | 0 | 3 | 0.7 | −0.5, |
| | | | | | 0, +0.5 |
| 4B | 2.5 | 10 | 3 | 0.7 | −0.5, |
| | | | | | 0, +0.5 |
| 5B | 3 | 0 | 3 | 1.0 | −0.5, |
| | | | | | 0, +0.5 |
| 6B | 3 | 10 | 3 | 1.0 | −0.5, |
| | | | | | 0, +0.5 |
| 7B | 3.5 | 0 | 3 | 1.2 | −0.5, |
| | | | | | 0, +0.5 |
| 8B | 3.5 | 10 | 3 | 1.2 | −0.5, |
| | | | | | 0, +0.5 |

TABLE 3
Applicator embodiments with two lateral channels for receiving
a brachytherapy needle, with a first lateral channel for
receiving brachytherapy needle having 0 degree lateral
angle and second lateral channel for receiving brachytherapy
needle having 10 degree lateral angle.

| | | | Number of | | |
|---|---|---|---|---|---|
| | | | Lateral | | |
| | | Lateral | Channels | | Longi- |
| | | Angle of | for | Radial | tudinal |
| | Diameter | Channel | Receiving | Distance | distance |
| | of the | for | Needle | From the | from the |
| | Applicator | Receiving | with | center of | middle of |
| Applicator | Body | Needle (a₂) | Identified | the ovoid | Ovoid |
| # | (cm) | (degrees) | Angle | (cm) | (cm) |
| 1C | 2 | 0 | 1 | 0.5 | +1.0 |
| | 2 | 10 | 1 | 0.5 | −1.0 |
| 2C | 2 | 0 | 1 | 0.5 | −1.0 |
| | 2 | 10 | 1 | 0.5 | +1.0 |
| 3C | 2.5 | 0 | 1 | 0.7 | +1.0 |
| | 2.5 | 10 | 1 | 0.7 | −1.0 |
| 4C | 2.5 | 0 | 1 | 0.7 | −1.0 |
| | 2.5 | 10 | 1 | 0.7 | +1.0 |
| 5C | 3.0 | 0 | 1 | 1.0 | +1.0 |
| | 3.0 | 10 | 1 | 1.0 | −1.0 |
| 6C | 3.0 | 0 | 1 | 1.0 | −1.0 |
| | 3.0 | 10 | 1 | 1.0 | +1.0 |
| 7C | 3.5 | 0 | 1 | 1.2 | +1.0 |
| | 3.5 | 10 | 1 | 1.2 | −1.0 |
| 8C | 3.5 | 0 | 1 | 1.2 | −1.0 |
| | 3.5 | 10 | 1 | 1.2 | +1.0 |

TABLE 4
Possible applicator embodiments with three lateral channels for receiving
a brachytherapy needle, with either: (1) two lateral channel for receiving
brachytherapy needle having 0 degree lateral angle and one lateral
channel for receiving brachytherapy needle having 10 degree lateral
angle; or (2) two lateral channel for receiving brachytherapy needle
having 10 degree lateral angle and one lateral channel for receiving
brachytherapy needle having 0 degree lateral angle.

| | | | Number of | | |
|---|---|---|---|---|---|
| | | | Lateral | | |
| | | Lateral | Channels | | Longi- |
| | | Angle of | for | Radial | tudinal |
| | Diameter | Channel | Receiving | Distance | distance |
| | of the | for | Needle | From the | from the |
| | Applicator | Receiving | with | center of | middle of |
| Applicator | Body | Needle (a₂) | Identified | the ovoid | Ovoid |
| # | (cm) | (degrees) | Angle | (cm) | (cm) |
| 1D | 2 | 0 | 2 | 0.5 | +1.0, |
| | | | | | −1.0 |
| | 2 | 10 | 1 | 0.5 | 0.0 |
| 2D | 2 | 0 | 1 | 0.5 | 0.0 |
| | 2 | 10 | 2 | 0.5 | +1.0, |
| | | | | | −1.0 |
| 3D | 2.5 | 0 | 2 | 0.7 | +1.0, |
| | | | | | −1.0 |
| | 2.5 | 10 | 1 | 0.7 | 0.0 |
| 4D | 2.5 | 0 | 1 | 0.7 | 0.0 |
| | 2.5 | 10 | 2 | 0.7 | +1.0, |
| | | | | | −1.0 |
| 5D | 3.0 | 0 | 2 | 1.0 | +1.0, |
| | | | | | −1.0 |
| | 3.0 | 10 | 1 | 1.0 | 0.0 |
| 6D | 3.0 | 0 | 1 | 1.0 | 0.0 |
| | 3.0 | 10 | 2 | 1.0 | +1.0, |
| | | | | | −1.0 |
| 7D | 3.5 | 0 | 2 | 1.2 | +1.0, |
| | | | | | −1.0 |
| | 3.5 | 10 | 1 | 1.2 | 0.0 |
| 8D | 3.5 | 0 | 1 | 1.2 | 0.0 |
| | 3.5 | 10 | 2 | 1.2 | +1.0, |
| | | | | | −1.0 |

Referring now to FIGS. 1-13, the various applicator 10, 100, 200, 300 disclosed herein can be utilized in combination with the conduit 5 to provide different intracavitary assemblies to accommodate different individual anatomies and/or address different target treatment areas with brachytherapy needles.

As the applicators 10, 100, 200, 300 are adapted to be removably secured to the conduit 5, multiple applicators 10, 100, 200, 300 made in accordance with the present disclosure can be interchanged with a single conduit 5. Accordingly, in another aspect, the present disclosure also includes intracavitary applicator kits for radiation oncology, which include a conduit 5 and two or more applicators 10, 100, 200, 300 that are made in accordance with the present disclosure and can be selectively removably secured to the conduit 5. The ability for a physicians to selectively choose amongst multiple applicators can provide several clinically meaningful advantages that can improve the quality and effectiveness of patient care. For instance different anatomical sites, tumor geometries, treatment areas and/or treatment depths may require distinct radiation dose distributions in order to achieve the desired therapeutic effect while limiting exposure to surrounding healthy tissue. Additionally, the anatomical environment in which an applicator is disposed during brachytherapy treatment can vary across different individuals. By enabling physicians to interchangeably select between different applicators with different geometries and/or channel arrangement, radiation delivery to the specific anatomical and clinical requirements can be improved. The ability for a physicians to selectively choose amongst multiple applicators may also serve to streamline clinical workflows and/or costs by eliminating the need for multiple, standalone systems or machinery and/or complex manual adjustments to a device.

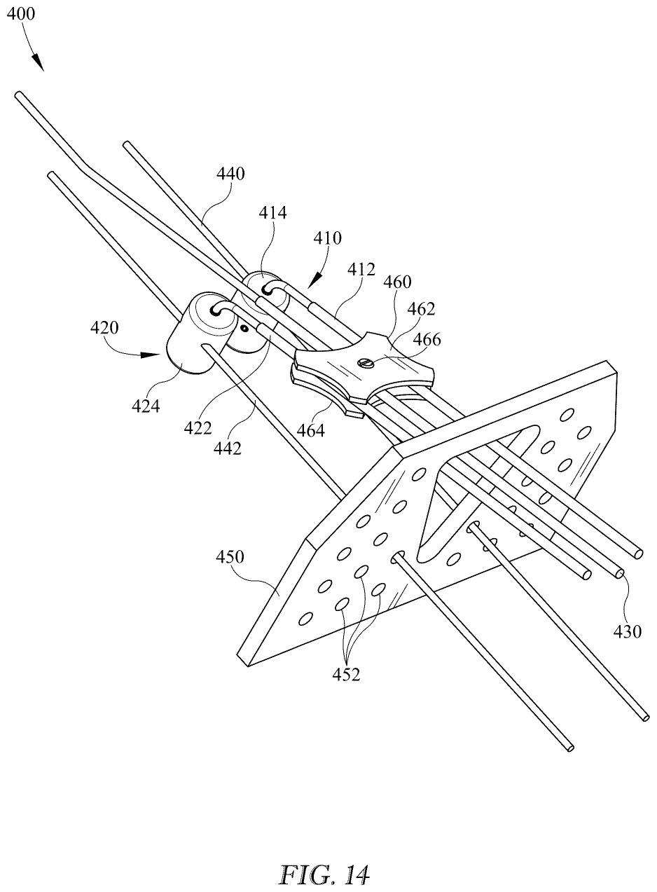

Multiple applicator assemblies made in accordance with the present disclosure and including various applicator 10, 100, 200, 300 embodiments disclosed herein can, in some embodiments and implementations, be utilized in a larger intracavitary applicator assembly (or assembly) 400 for radiation oncology. In this regard, and referring now to FIG. 14, an exemplary assembly 400 is provided and includes: a first intracavitary applicator assembly 410 configured to receive one or more brachytherapy needles 440; a second intracavitary applicator assembly 420 configured to receive one or more brachytherapy needles 442; a tandem applicator 430; a template 450; and a locking device 460.

Referring still to FIG. 14, the first intracavitary applicator assembly 410 and the second intracavitary applicator assembly 420 are each of the same general construction as the intracavitary applicator assembly 1 described above with reference to FIGS. 1-4. In this regard, the first intracavitary applicator assembly 410 and the second intracavitary applicator assembly 420 each include a conduit 412, 422 for receiving and guiding a radiation source and an intracavitary applicator 414, 424 removably secured to the conduit 412, 422. The intracavitary applicator 414, 424 of the first intracavitary applicator assembly 410 and the second intracavitary applicator assembly 420 can be any of the various intracavitary applicator 10, 100, 200, 300 described above with reference to FIGS. 1-13.

Referring still to FIG. 14, the tandem applicator 430 is configured to be inserted into a cervical canal of an individual and to receive and guide the radiation source from an open proximal end of the tandem applicator toward a closed distal end of the tandem applicator. In this regard, the tandem applicator 430 includes an elongated body with an internal channel along which the radiation source can travel along the length of the tandem applicator 430. In some embodiments, a portion of the tandem applicator 430, such as its distal end, may be curved or bent to facilitate insertion and/or treatment. It is appreciated that the position of the tandem applicator 430 relative to the intracavitary applicators 414, 424 can be modified compared to that shown in FIG. 14 without departing from the spirit and scope of the present disclosure. For instance, in some embodiments, the tandem applicator 430 may be lowered as to be positioned between the applicator bodies 414, 414 of the intracavitary applicators 414, 424. In some embodiments, the locking device 460 is configured to maintain the tandem applicator 430 in such position. The template 450 configured to support a proximal end of the one or more brachytherapy needles 440 received by the first intracavitary applicator assembly 410 and the one or more brachytherapy needles 442 received by the second intracavitary applicator assembly 420. In this regard, the template 450 includes a plurality of openings 452 through which brachytherapy needles 440, 442 can be inserted. In some embodiments, some or all of the plurality of openings may also be configured to receive a portion of the conduits 412, 422 of the first intracavitary applicator assembly 410 and the second intracavitary applicator assembly 420 and/or the tandem applicator 430.

Referring still to FIG. 14, in this exemplary embodiment, the locking device 460 is in the form of a clamp-style fastener that receives and maintains the conduits 412, 422 of the first intracavitary applicator assembly 410 and the second intracavitary applicator assembly 420 in a fixed position by applying a clamping force thereto. The locking device 460 can thus serve to couple the conduits 412, 422 of the first intracavitary applicator assembly 410 and the second intracavitary applicator assembly 420. The locking device 460 includes a pair of opposing plates 462, 464 between which the conduits 412, 422 of the first intracavitary applicator assembly 410 and the second intracavitary applicator assembly 420 are deposited, and a screw 466. The screw 466 can be selectively tightened to draw the opposing plates 462, 464 closer together to hold the conduits 412, 422 of the first intracavitary applicator assembly 410 and the second intracavitary applicator assembly 420 in a fixed position. Conversely, the screw 466 can be loosened to move the opposing plates 462, 464 further apart to permit rotation and/or sliding of the conduits 412, 422 of the first intracavitary applicator assembly 410 and the second intracavitary applicator assembly 420. In alternative embodiments, other locking devices that can be selectively manipulated by a user to transition the locking device between a first state that holds the conduits 412, 422 of the first intracavitary applicator assembly 410 and the second intracavitary applicator assembly 420 in a fixed position and a second state that permits rotation and/or sliding of the conduits 412, 422 of the first intracavitary applicator assembly 410 and the second intracavitary applicator assembly 420 can be utilized without departing from the spirit and scope of the present disclosure. In this embodiment, the locking device 460 is further configured to receive and maintain the tandem applicator 430 in a fixed position. Accordingly, in this embodiment, the locking device 460 serves to couple the tandem applicator 430 to the conduits 412, 422 of both the first intracavitary applicator assembly 410 and the second intracavitary applicator assembly 420. In alternative embodiments, the tandem applicator may 430 be coupled to only one of the conduits 412, 422 of the first intracavitary applicator assembly 410 and the second intracavitary applicator assembly 420.

Referring still to FIG. 14, in use, the assembly 400 can be utilized to apply brachytherapy treatment for cancer by depositing the applicator body of the first intracavitary applicator assembly 410 into a first vaginal fornix of an individual, depositing the applicator body of the second intracavitary applicator assembly 420 into a second vaginal fornix of the individual, and depositing a portion of the tandem applicator 430, in a uterine cavity of the individual. The intracavitary applicator 414, 424 of one or both the first intracavitary applicator assembly 410 and the second intracavitary applicator assembly 420 can be loaded with one or more brachytherapy needles 440, 442. Once the first intracavitary applicator assembly 410 and the second intracavitary applicator assembly 420 and any brachytherapy needles 440, 442 being utilized in treatment are in place, a radiation source (not shown) can be sequentially inserted into the internal channels of the first intracavitary applicator assembly 410, the second intracavitary applicator assembly 420, the tandem applicator 430, and any brachytherapy needles 440, 442 to apply the desired radiation dosing.

Although the assembly 400 is primarily described herein as including both the tandem applicator 430 and the template 450. Alternative embodiments in which the tandem applicator 430 and/or the template 450 are omitted from the assembly 400 are also contemplated herein.

Radiation sources that can be utilized in combination with the various applicator 10, 100, 200, 300 and assembly 1, 400 embodiments disclosed herein include, by way of non-limiting example, iridium-192 (Ir-192), cesium-137 (Cs-137), or cobalt-60 (Co-60) seeds that, in some cases, may be carried by a flexible wire, catheter, needle, or other carrying device suitable for insertion into the internal channels of the applicator 10, 100, 200, 300 or assembly 1, 400. In some embodiments and implementations, a radiation source loaded into the applicator 10, 100, 200, 300 or assembly 1, 400 may be arranged in the form of a train of radiation seeds. Loading techniques which may be employed to load a radiation source into the various embodiments of the applicators 10, 100, 200, 300 and assemblies 1, 400 disclosed herein are readily known within the art.

Although the applicators of the present disclosure are primarily described herein as including both a lateral channel for receiving a fastener and one or more lateral channels for receiving one or more brachytherapy needles, alternative applicator embodiments which include only one of: (i) a lateral channel for receiving a fastener; and (ii) one or more lateral channels for receiving brachytherapy needles are also contemplated.

In some embodiments and implementations, a radiation source may be loaded into the various embodiments of the applicators 10, 100, 200, 300 and assemblies 1, 400 disclosed herein via an afterloading machine, which, in some embodiments and implementations, may be computer-controlled. In some embodiments and implementations, an image guidance system, such as a computed tomography (CT) system, magnetic resonance imaging (MRI) system, and/or ultrasound system may be employed to facilitate guidance of the applicators 10, 100, 200, 300 and assemblies 1, 400 to a desired location within an individual in need of brachytherapy. Treatment planning software can also be utilized in the treatment of an individual in need of brachytherapy to assist in the calculation of dose distribution and optimized source positioning for treatment of an individual.

Accordingly, the various applicator 10, 100, 200, 300 and assembly 1, 400 embodiments disclosed herein can be utilized as part of a larger system that includes one or more of: an afterloading machine that assists in the deposit of a radiation source into the internal channels of the applicator 10, 100, 200, 300 or assembly 1, 400; an image guidance system; and/or treatment planning software stored in a computer-readable medium and executed by one or more processors.

The various components of the applicator embodiments disclosed herein are preferably constructed of medical-grade materials. In this regard, and in some embodiments, the materials utilized to construct the components of the various applicator 10, 100, 200, 300 and assembly 1, 400 embodiments comply with International Organization Standardization (ISO) 10993. In various embodiments, the conduits 5, 412, 422, the tandem applicator 430, and the locking device 460 may be constructed, in whole or in part, of a metal material, such as, by way of non-limiting example, titanium, stainless steel, cobalt-chromium, tantalum, platinum, magnesium, and combinations thereof. In some embodiments, the conduits 5, 412, 422, the tandem applicator 430, and the locking device 460 are constructed of titanium. In various embodiments, the applicator bodies 20, 120, 220, 320, of the applicators 10, 100, 200, 300, 414, 424, brachytherapy needles 40, 440, 442, and template 450 may be constructed, in whole or in part, of a plastic material, such as, by way of non-limiting example, acetal copolymer (POM-C), acrylic (PMMA), polycarbonate, polyether ether ketone (PEEK), polypropylene (PP), polyphenylsulfone (PPSU), ultra-high molecular weight polyethylene, polytetrafluoroethylene (PTFE), polysulfone (PSU), polyphenylene sulfide (PPS), polyvinylidene fluoride (PVDF), polyetherimide (PEI), polyphenylene oxide (PPO), derlin, plastics from FormLabs® BioMed series of compatible plastics, and combinations thereof. In some embodiments, some or all the components of the applicator 10, 100, 200, 300 or assembly 1, 400 may be constructed, in whole or in part, of a radiopaque material or combination of radiopaque materials.

Although the applicators 10, 100, 200, 300 and assemblies 1, 400 are sometimes disclosed herein in the context of being utilized for the treatment of a particular type of cancer, it is appreciated that the disclosed applicators 10, 100, 200, 300 and assemblies 1, 400 may find utility in the treatment of other types of cancer. In various implementations, the applicators 10, 100, 200, 300, 400 disclosed herein may be utilized in high-dose rate or low-dose rate brachytherapy treatments.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. An intracavitary applicator for radiation oncology, comprising:
an applicator body configured to be inserted in a vaginal fornix of an individual, the body including
a longitudinal channel configured to receive a conduit for receiving and guiding a radiation source into the longitudinal channel, and
a lateral channel providing passage from an exterior of the applicator body to the longitudinal channel; and
a fastener movably received in the lateral channel, such that the fastener can be selectively manipulated in a first manner to maintain the conduit in association with the applicator body when the conduit is received in the longitudinal channel, and in a second manner to permit removal of the conduit from the longitudinal channel.

2. The intracavitary applicator of claim 1, wherein the fastener is removably received in the lateral channel.

3. The intracavitary applicator of claim 1, wherein an interior surface of the applicator body defining the lateral channel includes threading, and wherein an exterior surface of the fastener includes threading compatible with the threading of the interior surface of the applicator body, such that the position of the fastener in the lateral channel can be adjusted by rotation of the fastener.

4. The intracavitary applicator of claim 1, wherein the fastener has a length that is greater than a length of the lateral channel.

5. The intracavitary applicator of claim 1, wherein the applicator body further includes an additional lateral channel, the additional lateral channel being configured to receive and guide a brachytherapy needle through the applicator body.

6. The intracavitary applicator of claim 5, wherein a centerline of the additional lateral channel is angled relative to at least one of a horizontal plane bisecting a first open end of the additional lateral channel and a vertical plane along which a longitudinal axis of the body extends.

7. The intracavitary applicator of claim 6, wherein the centerline of the additional lateral channel intersects with at least one of the horizontal plane bisecting the first open end of the additional lateral channel at an angle of about 0.1 degree to about 15 degrees and the vertical plane along which the longitudinal axis of the body extends at an angle of about 0.1 degree to about 15 degrees.

8. The intracavitary applicator of claim 1, wherein the body includes two or more additional lateral channels, with each lateral channel of the two or more additional lateral channels configured to receive and guide the brachytherapy needle through the body.

9. The intracavitary applicator of claim 8, wherein each lateral channel of the two or more additional lateral channels is provided on the same side of a vertical plane along which a longitudinal axis of the applicator body extends.

10. The intracavitary applicator of claim 8, wherein a centerline of at least one lateral channel of the two or more additional lateral channels is angled relative to at least one of a horizontal plane bisecting a first open end of the at least one lateral channel of the two or more additional lateral channels and a vertical plane along which a longitudinal axis of the body extends.

11. The intracavitary applicator of claim 10, wherein the centerline of the at least one lateral channel of the two or more additional lateral channels intersects with at least one of (i) the horizontal plane bisecting the first open end of the at least one lateral channel of the two or more additional lateral channels at an angle of about 0.1 degree to about 15 degrees and (ii) the longitudinal plane along which the longitudinal axis of the body extends at an angle of about 0.1 degree to about 15 degrees.

12. The intracavitary applicator of claim 1, wherein the applicator body includes one or more curved surfaces.

13. An intracavitary applicator assembly for radiation oncology, comprising:
(a) a first intracavitary applicator, including
(i) a first applicator body configured to be inserted in a first vaginal fornix of an individual, the first applicator body including
a longitudinal channel, and
a lateral channel providing passage from an exterior of the first applicator body to the longitudinal channel of the first applicator body, and
(ii) a first fastener movably received in the lateral channel of the first applicator body;
(b) a first conduit removably secured to the first applicator body by the first fastener, the first conduit being configured to receive and guide a radiation source into the longitudinal channel of the first applicator body;
(c) a second intracavitary applicator, including
(i) a second applicator body configured to be inserted in a second vaginal fornix of the individual, the second applicator body including
a longitudinal channel, and
a lateral channel providing passage from an exterior of the second applicator body to the longitudinal channel of the second applicator body, and
(ii) a second fastener movably received in the second lateral channel of the second applicator body; and
(d) a second conduit removably secured to the second applicator body by the second fastener and coupled to the first conduit, the second conduit being configured to receive and guide the radiation source into the longitudinal channel of the second applicator body.

14. The intracavitary applicator assembly of claim 13, and further comprising at least one of:
(e) a tandem applicator coupled to at least one of the first conduit and the second conduit, the tandem applicator configured to be inserted into a cervical canal of the individual and to receive and guide the radiation source from an open proximal end of the tandem applicator toward a closed distal end of the tandem applicator;
(f) a template coupled to at least one of the first conduit and the second conduit, the template including a plurality of openings through which a plurality of brachytherapy needles can be selectively inserted; and
(g) a locking device configured to be selectively manipulated to hold the first conduit and the second conduit in a fixed position.

15. The intracavitary applicator assembly of claim 13, wherein the first applicator body and the second applicator body each include an additional lateral channel, the additional lateral channel of the first applicator body and the second applicator body being configured to receive and guide a brachytherapy needle through the first applicator body or the second applicator body.

16. The intracavitary applicator assembly of claim 15, wherein a centerline of the additional lateral channel of the first applicator body and the second applicator body is angled relative to at least one of a horizontal plane bisecting a first open end of the additional lateral channel and a vertical plane along which a longitudinal axis of the first applicator body or the second applicator body extends.

17. The intracavitary applicator assembly of claim 16, wherein the centerline of the lateral channel intersects with at least one of the horizontal plane bisecting the first open end of the additional lateral channel at an angle of about 0.1 degree to about 15 degrees and the vertical plane along which the longitudinal axis of the first applicator body or the second applicator body extends at an angle of about 0.1 degree to about 15 degrees.

18. An intracavitary applicator kit for radiation oncology, comprising:
(a) a conduit for receiving and guiding a radiation source;
(b) a first intracavitary applicator configured to be removably secured to the conduit, the first intracavitary applicator including
(i) a first applicator body configured to be inserted in a vaginal fornix of an individual, the first applicator body including
a longitudinal channel configured to receive the conduit, and
a lateral channel providing passage from an exterior of the first applicator body to the longitudinal channel of the first applicator body, and
(ii) a first fastener movably received in the lateral channel of the first applicator body, such that the first fastener can be selectively manipulated in a first manner to maintain the conduit in association with the first applicator body when the conduit is received in the longitudinal channel of the first applicator body, and in a second manner to permit removal of the conduit from the longitudinal channel of the first applicator body; and
(c) a second intracavitary applicator configured to be removably secured to the conduit, the second intracavitary applicator including
(i) a second applicator body configured to be inserted in the vaginal fornix of the individual, the second applicator body including
a longitudinal channel, and
a lateral channel providing passage from an exterior of the second applicator body to the longitudinal channel of the second applicator body, and
(ii) a second fastener movably received in the lateral channel of the second applicator body, such that the second fastener can be selectively manipulated in a first manner to maintain the conduit in association with the second applicator body when the conduit is received in the longitudinal channel of the second applicator body, and in a second manner to permit removal of the conduit from the longitudinal channel of the second applicator body.

19. The intracavitary applicator kit of claim 18, wherein a diameter of the first applicator body is different from a diameter of the second applicator body.

20. The intracavitary applicator of claim 18, wherein the first applicator body and the second applicator body each include one or more additional lateral channels for receiving a brachytherapy needle, and wherein at least one of the number of additional lateral channels, the orientation of additional lateral channels, and the angle of additional lateral channels differs between the first applicator body and the second applicator body.
